(19) United States
(12) Patent Application Publication
(10) Pub. No.: US 2021/0346542 A1
(43) Pub. Date: Nov. 11, 2021

(54) ULTRAVIOLET GERMICIDAL IRRADIATION ROOM ANALYSIS

(71) Applicant: Cello Lighting, Inc., San Jose, CA (US)

(72) Inventor: Chenghung Pan, Palo Alto, CA (US)

(21) Appl. No.: 17/092,010

(22) Filed: Nov. 6, 2020

Related U.S. Application Data
(60) Provisional application No. 63/022,488, filed on May 9, 2020.

(51) Int. Cl. A61L 2/10

(52) U.S. Cl. CPC A61L 2/10; A61L 2202/12; A61L 2202/25

(57) ABSTRACT

An ultraviolet germicidal irradiation (UVGI) room analysis method and system combines the medical knowledge of UVGI disinfection and UVGI photometric analysis to generate a visual report that demonstrates the exposure time needed from an UVGI source at one or more locations within a room or other environment to effectively disinfect a specific microorganism, such as a germ or a virus, from each targeted surface in the room.

### CROSS-REFERENCE TO RELATED APPLICATIONS

This patent document claims benefit of the earlier filing date of U.S. provisional Pat. App. No. 63/022,488, entitled “An Ultraviolet Germicidal Irradiation Room Analysis,” filed May 9, 2020, which is hereby incorporated by reference in its entirety.

### BACKGROUND

Ultraviolet (UV) light is electromagnetic radiation with wavelengths shorter than visible light but longer than X-rays. UV light is generally categorized into several wavelength ranges, and short-wavelength UV light having wavelengths between about 200 nm and 300 nm, sometimes referred to as UVC light, is considered to be “germicidal UV.” In particular, organic materials such as nucleic acids strongly absorb UV wavelengths between about 200 nm and 300 nm, and the energy absorbed in an organic organism such as bacteria or viruses can result in the death or inactivation of the organism.

The effectiveness of germicidal UV for killing a microorganism depends on the length of time the microorganism is exposed to UV radiation, the intensity and wavelength of the UV radiation, the amount of protective intervening material around the microorganism, and the microorganism's ability to withstand UV exposure. In a germicidal system having a UV light source, the effectiveness of disinfection using the UV light source also depends on the line-of-sight distance between the microorganisms and the UV light source. Efficient disinfection using an ultra-violet germicidal irradiation (UVGI) system thus depends on the characteristics of the UVGI system and the placement of the UVGI system or particularly placement of the UV light source(s) and the line-of-sight to the location being disinfected. For project disinfecting a large area such as a room, the line of sight generally varies significantly at locations throughout the room or other large area being disinfected. Accordingly, a user operating a UVGI to disinfect a room, often does not know whether the disinfection is effective throughout the entire room.

### BRIEF DESCRIPTION OF THE DRAWINGS

FIG. 1 is a flow diagram of a process for generating a UV photometric analysis report in accordance with one example of the present disclosure.

FIG. 2 illustrates system in accordance with an example for performing a UV photometric analysis for sterilizing a target environment.

FIG. 3 illustrates an example analysis result indicating the effects of an ultraviolet germicidal irradiation system on the floor and a wall of a target environment being disinfected.

FIG. 4 shows a perspective illustration based on a three-dimensional model or data structure of a room for VIRA analysis in accordance with one example of the present disclosure.

FIG. 5 shows a perspective illustration based on a three-dimensional with color or grayscale coding of required exposure times to disinfect surfaces in the room of FIG. 4.

FIGS. 6-1, 6-2, and 6-3 show required exposure time report graphics resulting from analysis based on a UV source being respectively at a first position within a room, a second position within the room, and the two positions sequentially or simultaneously.

FIG. 7 illustrates monochrome patterns that may be used instead of colors or grayscale in graphical reports in accordance with an example of the present disclosure.

The drawings illustrate examples for the purpose of explanation and are not of the invention itself. Use of the same reference symbols in different figures indicates similar or identical items.

### DETAILED DESCRIPTION

A user operating an ultra-violet germicidal irradiation (UVGI) system to disinfect a room, often does not know whether or not the disinfection is effective at every location in the room. One way to evaluate the effectiveness of UVGI at one specific location in a room is to place an ultraviolet (UV) dosimeter at the location and measure the intensity of UVGI (e.g., μW/cm²) or a dose per area (e.g., μWs/cm²) during an exposure time at the location. The measured UV dose per area indicates the accumulated amount of germicidal UV energy that a microbial population in an area would absorb during the exposure time, and therefore indicates what percentage of the microbes that the exposure would be killed or deactivated. However, measuring the intensity or dose per area at every location (or even a sufficient number of locations) within a room or large area is time-consuming and may be impractical. For example, a measurement process may need to place a UV dosimeter at locations with inch or closer spacing on every surface in the room, which is impractical in a typical room or workspace, e.g., 1000 sq. ft. area including a floor, walls, ceiling, and furnishings.

The UV effectiveness in air and surface disinfection applications may alternatively be determined or estimated by calculating UV doses delivered to microbial populations at any desired number of locations. For example, a UV dose per area in μWs/m² or poules/m² at a single location may be calculated or estimated using Equation 1. UV lamps are typically rated or may be calibrated to provide known UV intensity ratings that the lamps nominally provide at a standard distance. For example, a UV intensity rating of a lamp may have units of power per area, e.g., μWs/cm² at a distance of 1 meter. In Equation 1, the UV intensity for a location at a given distance from a lamp differs from the UV intensity rating of the lamp by a distance adjustment factor, e.g., inverse square law, unless the distance to the lamp is exactly the standard distance, e.g., 1 m.

UV dose per area=UV intensity (μW/m²)×exposure time (seconds) Equation 1:

In accordance with an aspect of the current disclosure, a process or system uses calculated UV intensities, and required exposure times to achieve target levels of disinfection and provides reports with intuitive presentations enabling operators to complete effective disinfection of a room or environment with minimum time, energy, or effort using a UVGI system. In accordance with an example of the present disclosure, a report may provide effectiveness values (e.g., percentage disinfection) and/or required exposure times for locations throughout a 3D environment, e.g., a room or workspace. An ultra-violet germicidal irradiation (UVGI) analysis method or system can particularly evaluate a room or other environment and determine the required exposure times that a UVGI system needs to achieve effective UVGI disinfection of the room or environment.

In accordance with an aspect of the present disclosure, a Viral Irradiation Room Analysis (VIRA) process is used for the purpose of UV disinfection, which means for medical purposes, not lighting purposes. A goal of the VIRA process is to provide a guideline for disinfection operators to complete disinfection efficiently and effectively. Consequently, on top of room dimensions and UVGI source information, VIRA considers the medical goal of UV dosage required to inactivate a specific type of microbes.

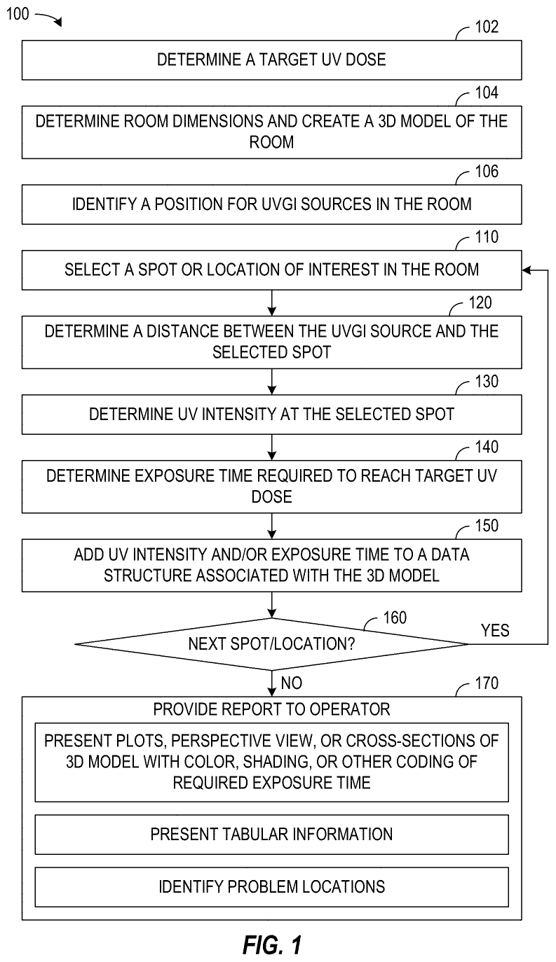

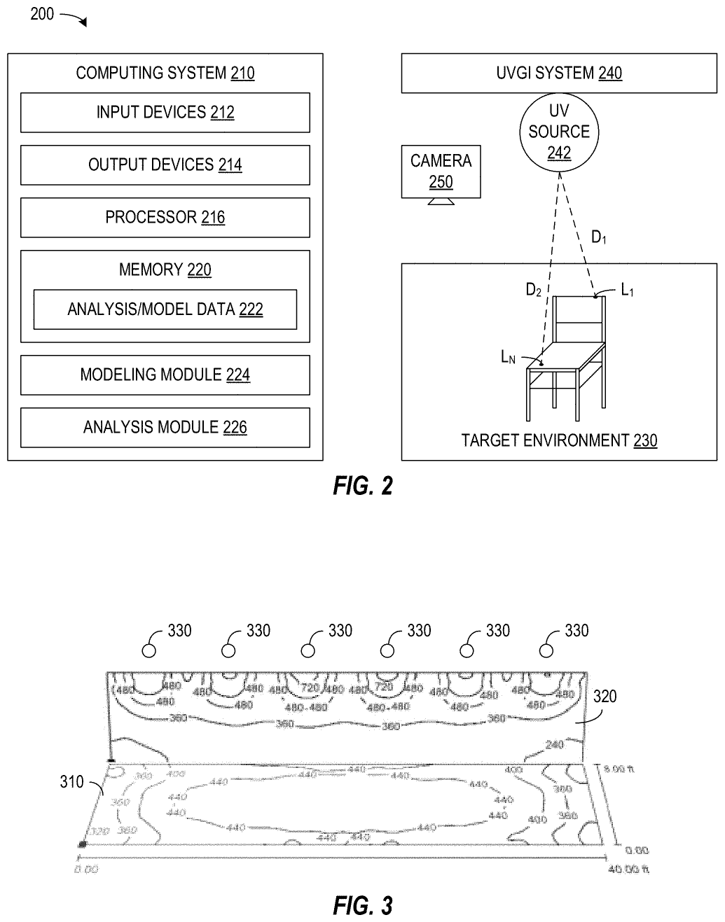

FIG. 1 is a flow diagram of a process 100 for evaluating the effectiveness of a disinfection process. Process 100 may be conducted using a computer or other general or special purpose processing hardware with suitable data, software, and/or firmware for implementing process 100. FIG. 2, for example, shows a block diagram of a system 200 providing hardware that may implement process 100. System 200 particularly includes a computing system 210 configured to analyze a process for disinfecting an environment 230 using a UVGI system 240. Computing system 210 may, for example, include one or more computer, e.g., one or more conventional servers, desktop computers, laptop computers, tablets, or smart phones, that collectively include input and output devices 212 and 214, one or more processors 216, and memory 220. Input and output devices 212 and 214 receive or transmit data to or from other devices, receive user commands or data input, and implement a user interface to receive user input, e.g., commands or data, and provide the user with a report for the process of disinfecting environment 230.

Process 100 of FIG. 1 includes the initial process blocks 102, 104, and 106 that respectively determine a target UV dose for disinfection of selected microbes from a room or environment, e.g., target environment 230 of FIG. 2, determine a three-dimensional model of the room or other environment to be disinfected, and identify positions of one or more UVGI sources in the room or other target environment. The following describes process 100 of FIG. 1 with reference to the example of system 200 disinfecting target environment 230 of FIG. 2, but as will be apparent, disclosed processes may be applied for disinfecting any environments, e.g., surfaces of specific items or fixtures or room interiors, work areas, or exterior locations.

Process block 102 includes determining a target UV dose for effective disinfection. The effectiveness of the UVGI, however, may depend on the kind of microbe(s) being targeted and a level of eradication required or desired. A target doses of germicidal UV that provides the desire level of eradiation for particular microbes may be known in the literature or determined from studies of the microbes. In particular, each kind of microbe, including viruses and germs, may have a unique dosage table that indicates the minimum germicidal UV dose required to eradicate a specific percentage, such as 90% (1 log), 99% (2 log), 99.9% (3 log) and so on, of the target microbes. For example, Table 1 shows that experimentally determined UV doses or fluences needed to cause a log 1 (90%) reduction, log 2 (99%) reduction and log 3 (99.9%) reduction of Aspergillus brasiliensis and Bacillus subtilis. The target dose determined in process block 102 needs to be applied throughout an environment to be disinfected.

TABLE 1
Fluences for multiple log reductions for various spores

| | Lamp | Fluence (mJ/cm²) needed for given log reduction | | |
|---|---|---|---|---|
| Microbe | Type | Log 1 (90%) | Log 2 (99%) | Log 3 (99.9%) |
| Aspergillus | LP | 122 | 226 | 293 |
| brasiliensis | | | | |
| Bacillus | LP | 20 | 39 | 60 |
| subtilis | | | | |

Process block 104 determines or constructs a 3D model of the target environment being disinfected. Process block 104 may particularly employ 3D modeling of room dimensions and furniture positioned inside the room, and the 3D model from block 104 may particularly represent surfaces, e.g., the surfaces of floors, walls, ceilings, room fixtures and furniture, within the room. In system 200 of FIG. 2, a modeling module 224 receives input, e.g., images or measurements of target environment 230, and constructions a representation of the target environment 230 using model data 222. The 3D model may be based on data including measured or otherwise known information about the target environment. Modeling module 224 may, for example, receive image data from a camera 250 that images target environment 230 from multiple view points, and modeling module 224 may initially construct model data 222 from the image data and other data that a user may provide regarding target environment 230. Room information may indicate the shape and dimensions of the room, the shape and dimensions of any fixtures or furniture in the room, and the staging or positioning of any movable or mobile furniture or fixtures inside the room, and process block 104 may be use any available data to create a 3D model of the room. The 3D model, e.g., model data 222, may, for example, be a polygonal model or polygon mesh, which is a common form of 3D model currently used in the animation, film, and game industries.

Process block 106 characterizes the UVGI system, e.g., UVGI system 240 in FIG. 2, and particularly identifies where within the 3D model space the one or more UVGI sources, e.g., source 242, will be during a disinfection process. The positions of UVGI sources may be determined using the specifications of each UVGI source, including the shape, intensity, or rating of the UVGI source, and a location of the UVGI source, e.g., relative to target environment 230 and represented in the 3D model. In some applications, the UVGI sources may be in fixtures that may be attached to the ceiling or walls of an environment such as a room. In some other applications, the UVGI sources may be portable or mobile and have one or more locations chosen for convenience of an operator or chosen through evaluation identifying most effective locations for the UVGI sources. The characterization data or information process block 106 uses for the UVGI system may include calibration data or the specifications of UVGI source 242, including the shape and intensity of UVGI source 242.

After the minimum dose level to inactivate the target microbes to some desired percentage and the locations of UVGI sources are determined (block 102), the effectiveness of UVGI disinfection may be determined at the locations of interest, e.g., on surfaces (as represented in the 3D model) that may harbor the targeted microbes. Process block 110, for example of system 200 in FIG. 2, represents selecting a current location L₁ of interest in the target environment 230, and process block 120 represents determining a line of sight distance D₁ (or distances) from the selected location L₁ to the location(s) of the UVGI source(s) 242. Based on the intensity rating of each UVGI source 242 and the distance from the UVGI source 242 to the selected location L₁, process block 130 may determine the UV intensity at the selected location L₁, and a process block 140 may determine a required exposure time (RET) needed for the UVGI source(s) to apply the target UV dose. Equation 2 is a formula for determining the required exposure time using a single UVGI source.

RET (seconds)=Target UV dose (mWs/cm²)/UV intensity (mW/cm²) Equation 2:

A process block 150 may store the UV intensity and the Required Exposure Time (RET) for a location in a data structure that is indexed by location and that represents the UV intensity or the RET. In system 200 of FIG. 2, analysis/model data may be updated to provide a representation of UV intensity and RET as function of location within the 3D model. After storing analysis information for a location, a decision block 160 in process 100 of FIG. 1 can determine whether any other locations, e.g., L₂ in FIG. 2, need to be evaluated in the target environment to being disinfected. If so, process 100 of FIG. 1 branches back to process block 110, which selects a next spot or location in the target environment. In some examples of process block 110, locations to be analyzed are distributed across all or selected areas of the surfaces defined by the 3D model of the target environment, and may be spaced apart with a relatively uniform spacing, e.g., locations may be spaced 1, 5, or 10 cm apart. Following each repetition of block 110 selecting a next location to be analyzed, process blocks 120, 130, 140, and 150 determine the line-of-sight distance(s) to the selected location, calculate the UV intensity or RET for the selected location, and store calculated UV intensity or RET in the data structure representing the 3D model. Process 100 may continue looping from decision block 160 to process block 110 until decision block 160 determines that all locations of interest in the target environment have been analyzed and have UV intensities or RETs stored in the data structure. In one specific example, the data structure can include a representation of a color value for each location of interest in the 3D model, e.g., a color 3D model may have surfaces with colors or textures indicating data values such as RETs at the locations of interest and may have colors interpolated based on the nearest locations of interest on the surfaces of the 3D model. The textures of a 3D model may alternatively use other methods for representing RETs, e.g., instead of color using grayscale shades, monochrome patterns, or contour lines.

A process block 170 provides an analysis report to an operator of a UVGI system to guide the operator in disinfecting the target environment. In one example of the present disclosure, the report provides RET information to the user in a graph or illustration form, e.g., on display device, e.g., one of output devices 214 in system 200 of FIG. 2. FIG. 3 illustrates a report may include an image showing surfaces with contour plots 310 and 320 representing UV intensity or RET for disinfection respectively. In FIG. 3, the illustrated surfaces include a floor and a wall of a room targeted for disinfection. In general, the patterns of plots 310 may result from using a UVGI system including a set of UVGI sources 330 that may be mounted in a sealing of the targeted room. Reporting block 170 may further include presenting information in a tabular or written form and/or may further identify problem locations, e.g., locations in the target environment with low UV exposure or long RETs.

Process block 106 and subsequent process blocks 110 to 160 may be repeated for one or more alternative positions of the UVGI source or sources in the target room or environment. Alternative positions of UVGI sources may be possible, for example, when a room or other environment is being disinfected using a mobile UVGI machine. In many situations, a user may want to determine where the UVGI source or sources should be position for efficient disinfection of the target environment. For each alternative locations of the UV source, process 100 can determine required exposure times at all spots of interest in the environment. In general, the duration of a disinfection process when the UV source is at a particular location needs longer than the maximum of the required exposure times for the spots of interest. An optimal location for the UV source may be selected to be the location that minimizes the maximum of the required exposure times for the spots of interest.

In accordance with an aspect of the current disclosure, the RETs are important parameters for disinfection operators disinfecting a target environment. In particular, when an operator enters a room, the operator may use the RET for a location to decide how long a UVGI machine should operate to irradiate the location and make sure the disinfection is effective at the selected location. However, it is time-consuming and impractical for an operator to place and operate a UVGI source to separately disinfect every surface location in a room. In accordance with an aspect of the current disclosure, a process, sometimes referred to herein as Viral Irradiation Room Analysis (VIRA), generates easy-to-understand guidelines or reports for disinfection operators to follow when disinfecting a room or other environment.

In one example of the present disclosure, the UV intensity at every surface spot in a room can be predicted by the UV photometric analysis process 100 of FIG. 1, and process block 170 can generate color coded or grayscale images of the room (or of the 3D model) from the 3D analysis/model data. UV intensity information may not be intuitive enough to enable an operator to easily and effectively disinfect a room because the UV disinfection operator may not know the goal for the UV dose and therefore may not know how long to irradiate the space being disinfected. Even if the operator knows the target dosage, e.g., 40,000μWs/cm², a presentation of UV intensity may still require the operator to calculate exposure times based on the intensity codes in a 3D model and the target dosage.

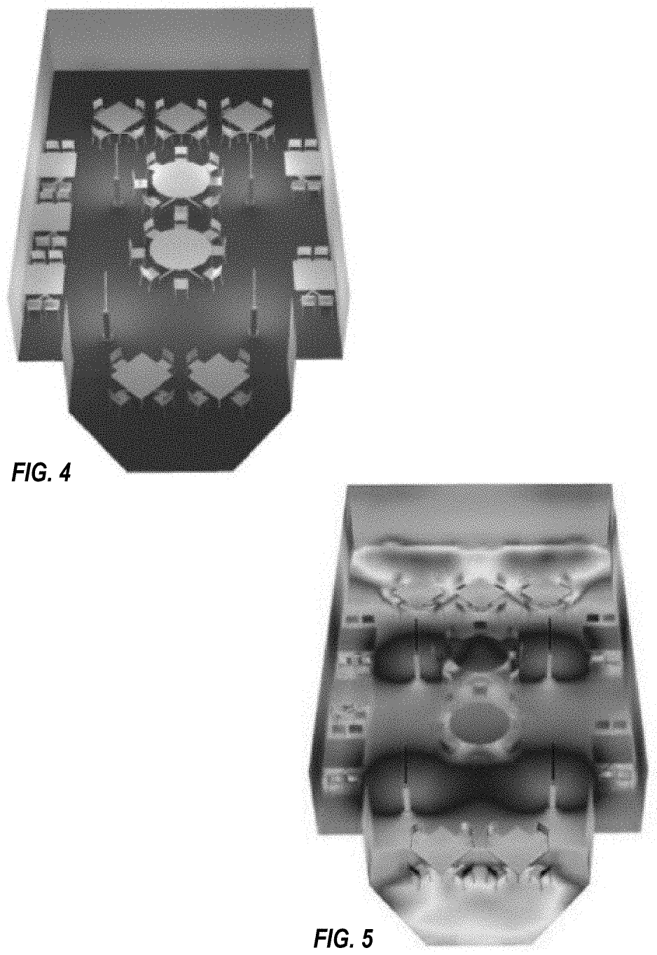

A report may provide an operator with a portion of a 3D model with coding, e.g., color or shading or pattern codes, representing the RETs. FIG. 4, for example, shows an example of a perspective view of surfaces represented in a 3D model of a dining area, and FIG. 5 show a view from the same perspective of the same surfaces with shading representing the required exposure time for disinfection of the surfaces. For example, in FIG. 5 which shades corresponding to red, orange, green, and bluish tending to black may be used for spots respectively requiring 1 minute, 5 minutes, 10 minutes, and 15 minutes or longer, to help the operator decide how long to wait before a room is effectively disinfect or to apply additional disinfection to areas that take too long of a time to disinfect.

VIRA analysis and reporting may not only be valuable for disinfection operators but also valuable for architects, facility managers, and for procurement managers. An architect may, for example, wish to analyze a virtual room or other architectural space to improve the design or select a UVGI system for the space. Facility or procurement managers may want to use VIRA analysis when budgeting purchase of UVGI machines based on time budgets for on-site applications. For instance, in a classroom, the disinfection time could be scheduled in the evening, when plenty of time is available and less UVGI machines are needed. Or the disinfection time could be scheduled in the recess time or lunch break, when the time is limited and more UVGI machines would be needed.

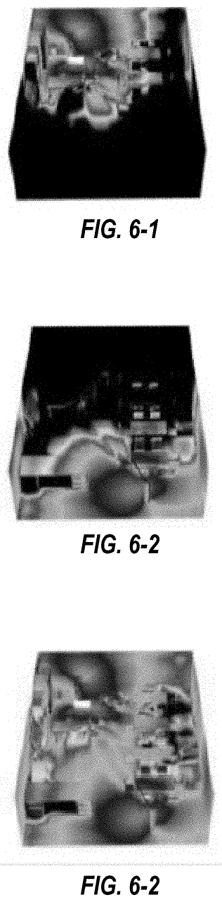

FIGS. 6-1, 6-2, and 6-3 illustrates VIRA results demonstrating the trade-offs between the number of UVGI machines and the RETs. FIG. 6-1 particularly shows a graphical display of required exposure times in a class room when a UVGI system is at a first location, and FIG. 6-2 shows a graphical display of required exposure times in the class room when a UVGI system is at a second location. Both FIGS. 6-1 and 6-2 show significant dark areas indicating portions of the classroom that have very long required exposure times when the UVGI system is in alternative locations. FIG. 6-3 shows that placing two UVGI system in the classroom at the same time or sequentially positioning a single UVGI system at the two locations can provide effective disinfection within an acceptable time.

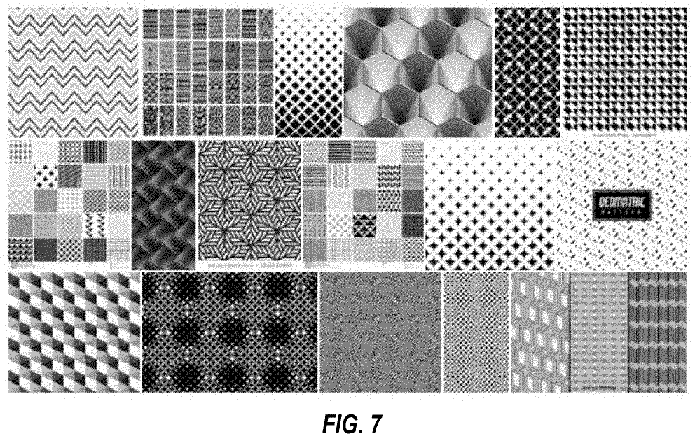

The RET graphical representation in VIRA results is not limited to color or grayscale patterns only. The RET results can also be represented by any monochrome pattern or texture as shown in FIG. 7 to help disinfection processes.

The RET representation in VIRA results also is not limited to graphic presentations. The RET results can also be represented in any text or tabular forms as exemplified here to help disinfection process. An example text or tabular forms is shown in the following Table 2. In Table 2, VIRA analysis of multiple rooms A001, B003, C006, and D005 is performed, and for each room a longest exposure time of any location of interest in the is determined for a specified location of a UVGI source. Table 2 presents that information, and indicates to an operator a location in each room to place the UVGI source and a minimum exposure time for effective disinfection of that room.

TABLE 2
Tabular VIRA Results Presentation

| Room ID | Location in the room | Required Exposure Time (RET) |
|---|---|---|
| A001 | Third row from front | 30 minutes |
| B003 | Northeast corner | 20 minutes |
| C006 | Second column from right | 60 minutes |
| D005 | 10 feet from the back wall | 40 minutes |

Each of the modules disclosed herein may include, for example, hardware devices including electronic circuitry for implementing the functionality described herein. In addition or as an alternative, each module may be partly or fully implemented by a processor executing instructions encoded on a machine-readable storage medium.

All or portions of some of the above-described systems and methods can be implemented in a computer-readable media, e.g., a non-transient media, such as an optical or magnetic disk, a memory card, or other solid state storage containing instructions that a computing device can read and execute to perform specific processes that are described herein. Such media may further be or be contained in a server or other device connected to a network such as the Internet that provides for the downloading of data and executable instructions.

Although particular implementations have been disclosed, these implementations are only examples and should not be taken as limitations. Various adaptations and combinations of features of the implementations disclosed are within the scope of the following claims.

What is claimed is:

1. A method for generating a disinfection guide for use of an ultraviolet germicidal irradiation (UVGI) machine to disinfect a target environment, the method comprising operating a processing hardware to perform a process including:
identifying a targeted UV dosage level for disinfection of the target environment;
determining a first location in the target environment for a UV source of the UVGI machine;
for each of a plurality of spots on surfaces in the target environment, determining a UV intensity that the UV source produces at the spot, and from the UV intensity, determining a required exposure time for the UV source at the first location to provide the target UV dosage level at the spot; and
from the required exposure times, generating the disinfection guide indicating a time required to disinfect the target environment.

2. The method of claim 1, further comprising the processing hardware constructing a 3D model of the target environment using dimensions of the target environment and data on any fixtures in the target environment, wherein generating the disinfection guide comprises using the 3D model to generate an image representing the surfaces in the target environment, wherein points in the image are marked to represent the required exposure times at the spots.

3. The method of claim 2, wherein constructing the 3D model comprises capturing images of the target environment including the fixtures.

4. The method of claim 2, wherein the image is marked so that points in the image respectively corresponding to the spots indicate the required exposure time at the spot, values of the exposure times being indicated by one of a color, a grayscale level, a graphic pattern, and a position relative to a contour plots in the image.

5. The method of claim 1, wherein identifying the target UV dosage comprises:
identifying a microbe;
identifying a desired reduction in the microbe in the target environment; and
identifying as the target UV dosage, a UV dosage found using a lookup table associated with the microbe.

6. The method of claim 1, wherein for each of the spots, determining the UV intensity that the UV source produces at the spot comprises determining the UV intensity from a distance between the spot and the first location of the UV source.

7. The method of claim 1, wherein generating the guide comprises generating a text or tabular of the required exposure times for the spots.

8. The method of claim 1, further comprising:
for each of one or more second locations in the target environment for the UV source, determining, for each of the spots on the surfaces of in the target environment, a UV intensity that the UV source in the second location produces at the spot, and from the UV intensity, determining a required exposure time that the UV source at the second location requires to provide the target UV dosage level at the spot; and
using the required exposure times to select which of the first and second locations to use to disinfect the target environment.

9. A non-transient media containing instructions that a computing device can read and execute to perform a process comprising:
identifying a targeted UV dosage level for disinfection of a target environment;
determining a location in the target environment for a UV source;
for each of a plurality of spots on surfaces of in the target environment, determining a UV intensity that the UV source produces at the spot, and from the UV intensity, determining a required exposure time for the UV source to provide the target UV dosage level at the spot; and
from the required exposure times, generating the disinfection guide indicating a time required to disinfect the target environment.

10. The non-transient media of claim 9, the process further comprises:
constructing a 3D model of the target environment using dimensions of the target environment and data on any fixtures in the target environment, wherein
generating the disinfection guide comprises using the 3D model to generate an image representing the surfaces in the target environment, points in the image being marked to represent the required exposure times at the spots.

11. The non-transient media of claim 10, wherein the image is marked so that points in the image respectively corresponding to the spots indicate the required exposure time at the spot, values of the exposure times being indicated by one of a color, a grayscale level, a graphic pattern, and a position relative to a contour plots in the image.

12. The non-transient media of claim 10, wherein the process further comprises:
for each of one or more second locations in the target environment for the UV source, determining, for each of the spots on the surfaces of in the target environment, a UV intensity that the UV source in the second location produces at the spot, and from the UV intensity, determining a required exposure time that the UV source at the second location requires to provide the target UV dosage level at the spot; and
using the required exposure times to select which of the first and second locations to use for the UV source during disinfection of the target environment.
